(19) United States
(12) Patent Application Publication
(10) Pub. No.: US 2007/0166372 A1
(43) Pub. Date: Jul. 19, 2007

(54) Preparation of solid coprecipitates of amorphous valsartan

(75) Inventors: Huimin He Huang, Northborough, MA (US); Caigu Huang, Northborough, MA (US)

Correspondence Address: Mai De Ltd., P. O. Box 193, Northborough, MA, 01776, US

(73) Assignee: Mai De Ltd.

(21) Appl. No.: 11/641,093

(22) Filed: Dec. 19, 2006

Related U.S. Application Data
(60) Provisional application No. 60/759,726, filed on Jan. 19, 2006.

(51) Int. Cl. A61K 31/4184; A61K 9/48; A61K 9/20; A61K 9/14

(52) U.S. Cl. 424/451; 424/464; 424/486; 514/394

(57) ABSTRACT

A novel coprecipitate of amorphous valsartan with a pharmaceutically acceptable carrier, e.g. polyvinylpyrolidone (PVP), crosslinked-polyvinylpyrolidone, polyvinylpyrolidone vinyl acetate copolymer (PVP-VA64), a process for the preparation of said novel co-precipitate and the use of said novel coprecipitate in the treatment and/or prophylaxis of hypertension, cardiovascular diseases and conditions associated with thereof and certain complications thereof, are disclosed. A novel solid solution of amorphous valsartan with a pharmaceutically acceptable carrier, preferably with polyethyelene glycol PEG from 4000 to 20,000 of average mol. wt., a process for the preparation thereof and use are disclosed. The said novel coprecipitate of amorphous valsartan and the said novel solid solution of valsartan are stable and may be particularly suitable for pharmaceutical dosage forms.

This application claims the benefit of priority to U.S. Provisional Patent Application Ser. No. 60/759,726, filed on Jan. 19, 2006, the entire disclosure of which is hereby incorporated by reference.

### FIELD OF THE INVENTION

This invention is related to new coprecipitate of amorphous valsartan with a pharmaceutically acceptable carrier, e.g. polyvinylpyrolidone (PVP), crosslinked-polyvinylpyrolidone, polyvinylpyrolidone vinyl acetate copolymer (PVP-VA64). The invention also provides a novel solid solution of valsartan with a pharmaceutically acceptable carrier, e.g. solid polyethyelene glycol (PEG). The invention further provides a process for the preparation of said co-precipitate and solid solution, and the use of said coprecipitate or solid solution in the treatment and/or prophylaxis of cardiovascular complaints such as hypertension and certain complications thereof. The said coprecipitate or solid solution of amorphous valsartan with a pharmaceutically acceptable inert carrier is stable and may be particularly suitable for bulk preparation and dosage form preparation.

### BACKGROUND OF THE INVENTION

Valsartan, its chemical name is (S)-N-(1-carboxy-2-methylpro-1-yl)-N-pentanoyl-N-[2′-(1H-tetrazol-5-yl)-biphenyl-4-yl-methyl]amine. Its chemical structure is as follows:

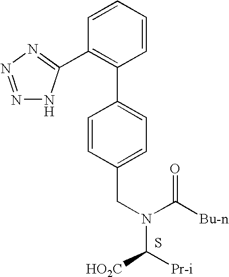

Valsartan belongs to the class of non-peptide angiotensin-II receptor antagonists having a very high selectivity for the AT₁ receptors. Valsartan is used as a single-substance preparation or in combination with the diuretic hydrochlorothiazide for the treatment of cardiovascular diseases such as hypertension, including high blood pressure. As AT₁ receptor antagonist, valsartan more particularly inhibits the blood pressure rise caused by angiotensin II, suppresses angiotensin-II-induced aldosteron secretion, and lowers angiotensin-II-induced liquid uptake.

U.S. patent (U.S. Pat. No. 5,399,578) discloses preparation of valsartan and its pharmaceutically acceptable salts, and its therapeutic use in the treatment of cardiovascular complaints such as hypertension. WO02/06253 discloses the preparation of various valsartan salts, including crystalline, partly crystalline, amorphous and polymorphous forms of specific salts of valsartan such as potassium salt, sodium salt, bis-diemethylammonium salt, calcium salt, magnesium salt and others.

It is known from the prior art that valsartan free acid mainly exists as an amorphous solid (cp. WO 02/06253), but can exist as well in partly crystalline forms or in a mixture of crystalline and amorphous material (cp. WO 02/06253; WO 03/089417; U.S. Pat. No. 05/0,222,233 and U.S. Pat. No. 04/0,242,661). The data reported in different patent documents for the melting points of valsartan differ considerably between reported values ranging from 80° C. to 117° C.

In WO 02/06253, it discloses amorphous form of valsartan: “The X-ray diffraction diagram consists essentially of a very broad, diffuse X-ray diffraction; the free acid is therefore characterized as almost amorphous under X-ray. The melting point linked with the measured melting enthalpy of 12 kJ/mol [approximately 28 j/g] unequivocally confirms the existence of a considerable residual arrangement in the particles or structural domains for the free acid valsartan”.

WO 03/089417 describes valsartan form I with melting points between 80° C. and 91° C. and valsartan form II with melting points between 91° C. and 102° C. Their X-ray powder diffraction (X-RPD) spectra indicated that most of the valsartan materials obtained were still amorphous valsartan. Truly pure crystalline Form I or Form II was not obtained in this publication or other prior arts.

Similarly, US2005/0222233 and US2004/0242661 describe preparation of crystalline forms (I-VIII, IX-XIII) and pure amorphous form of valsartan. The X-RPD spectra of the obtained crystalline materials indicated that most of the crystalline forms of valsartan were the mixtures of amorphous valsartan and crystalline valsartan. The crystallinity of the different forms of valsartan, when calculated according to crystalline function (by computing the ratio between the area of the crystalline peaks in the graph and the area of the whole graph) is as follows: Form I-62%, Form II-63%, Form III-35%, Form IV-48%, Form VI-40%, Form VII-42%, Form III-17% and Form IX-17%.

Although pure amorphous form (e.g., no melting point in DSC) of valsartan was obtained in this publication, it does not provide means to stabilize the pure amorphous valsartan. Pure amorphous valsartan is particularly not stable due to its inherent characteristic nature to exist in a mixture of amorphous and crystalline forms. Therefore pure amorphous valsartan itself is not suitable for direct use in preparation of pharmaceutical dosage form due to its polymorph instability and fluffy nature with low density.

Publication US2005/0234016 describes a process for the preparation of valsartan asorbates, wherein valsartan in a finely divided amorphous form adsorbs on crystalline materials such as lactose, mannitol and other excipients. Although this process produced the amorphous form of active ingredient and tried to make use of amorphous valsartan for pharmaceutical formulation, but stability of amorphous valsartan in such crystalline matrix was not evaluated for long-term storage. The disadvantage of this process and the adsorbates obtained thereof is that the crystalline adsorbing materials can induce the conversion of amorphous valsartan into crystalline valsartan during further processing or long-term storage, rendering it unsuitable for making stable solid pharmaceutical dosage forms.

The prior art indicated that the preparation of a pure crystalline form of valsartan appears very difficult, has never been achieved yet, and that a mixture of amorphous and crystalline materials of valsartan were usually obtained, regardless of processes used. The ratio of amorphous to crystalline in the mixture very strongly depends on process parameters or solvents, and different products will be obtained following slight changes in these parameters. In addition, the ratio of amorphous to crystalline in the mixture may change during storage or further processing, leading to the change in physiochemical properties (e.g., dissolution, bioavailability and stability) of active ingredient and drug product.

In order to prevent the conversion between amorphous form and crystalline form, and thus to control the quality of the active ingredient, a definitive control of polymorph form of active ingredient is required. The use of a mixture of polymorph forms, in particular, the use of a mixture of amorphous and crystalline forms should be undesirable in pharmaceutical formulations (e.g., solid dosage forms) since the crystalline material will readily induce the conversion of amorphous form into crystalline form, resulting in change of ratio of amorphous form to crystalline form, and thus change in solid properties.

Therefore, there are needs in stabilizing the polymorph form of valsartan, in particular, in stabilizing amorphous form of valsartan, in order to make stable pharmaceutical formulation of valsartan.

We have unexpectedly found that a novel stable coprecipitate or solid solution of amorphous valsartan solved this problem with an amorphous pharmaceutically acceptable carrier. Because of instability of amorphous form this may be overcome by preparing said novel coprecipitate or solid solution in order to stabilize the amorphous form of valsartan.

### SUMMARY OF THE INVENTION

In one aspect, the present invention relates to a new coprecipitate of amorphous valsartan with a pharmaceutically acceptable carrier. The said carrier is selected from polyvinylpyrolidone (PVP), crosslinked-polyvinylpyrolidone (cross-PVP) and polyvinylpyrolidone vinyl acetate copolymer (PVP-VA64).

In another aspect, the present invention relates to a novel solid solution of valsartan and its pharmaceutically acceptable carrier. The said carrier is solid polyethylene glycol (PEG).

In a further aspect, the present invention provides a process for the preparation of coprecipitate of amorphous valsartan with a pharmaceutically acceptable carrier, including the steps of dissolving crystalline valsartan in a suitable solvent(s) and removing the solvent(s) from the solution by a conventional technique to afford amorphous coprecipitate of valsartan with a pharmaceutical carrier. Such conventional techniques include, but are not limited to, distillation, distillation under reduced pressure or vacuum, evaporation and spray drying.

In a preferred aspect, the present invention relates to a process for preparing amorphous coprecipitate of valsartan with a pharmaceutical carrier, including the steps of dissolving valsartan in a suitable solvent(s) such as acetone methanol, ethanol to form a solution and distilling the solvent from the solution to afford said amorphous coprecipitate and then drying the product.

In a still aspect, the present invention relates to a process for preparing amorphous coprecipitate of valsartan and a pharmaceutical carrier, including the steps of dissolving valsartan and a pharmaceutical carrier in a suitable solvent(s) and removing the solvent(s) from the solution by distillation and spray drying.

In another aspect, the present invention relates to a process for preparing a novel solid solution of valsartan with solid polyethylene glycol (PEG), including the steps of dissolving valsartan and a carrier in a suitable solvent(s) and removing the solvent(s) from the solution by distillation and spray drying.

In a further aspect, the present invention relates to a process for preparing a novel solid solution of valsartan with solid polyethylene glycol (PEG), including the steps of melting valsartan and a pharmaceutically acceptable carrier to form a melt, and then cooling the obtained melted solution.

In still another aspect, the present invention provides a pharmaceutical formulation comprising coprecipitate of amorphous valsartan with a pharmaceutically acceptable carrier and other pharmaceutical excipients.

In further aspect, the present invention provides a pharmaceutical formulation comprising solid solution of amorphous valsartan with a pharmaceutically acceptable carrier and other pharmaceutical excipients.

According to a still aspect of the invention there is a method for prevention and treatment of cardiovascular complaints such as hypertension and heart failure, with a medicament made by using an effective amount of valsartan in a form of said coprecipitate or solid solution in unit dosage form.

### BRIEF DESCRIPTION OF THE ACCOMPANYING DRAWINGS

FIG. 1: X-ray powder diffraction pattern of a mixture of amorphous and crystalline valsartan.

FIG. 2: Differential scanning calorimetry (DSC) of a mixture of amorphous and Crystalline valsartan.

FIG. 3: X-ray powder diffraction pattern of amorphous valsartan with polyvinylpyrolidone (PVP).

FIG. 4: Differential scanning calorimetry (DSC) of amorphous valsartan with polyvinylpyrolidone (PVP).

FIG. 5: X-ray powder diffraction pattern of amorphous valsartan with polyvinylpyrolidone-vinyl acetate copolymer (PVP-VA64).

FIG. 6: Differential scanning calorimetry (DSC) of amorphous valsartan with polyvinylpyrolidone-vinyl acetate copolymer (PVP-VA64).

### DETAILED DESCRIPTION OF THE INVENTION

Unless otherwise indicated, the following definitions are set forth to illustrate and define the meaning and scope of the various terms used to describe the invention herein.

The term “crystalline” refers to a crystal or crystal modification that can be characterized by analytical methods such as X-ray powder diffraction, IR-spectroscopy, differential scanning calorimetry (DSC) or by its melting point.

The term “amorphous” means a solid without long-range crystalline order. Amorphous form of valsartan in accordance with the invention preferably contains less than about 20% crystalline forms of valsartan, preferably less than 10% crystalline form of valsartan, and more preferably less than 2% crystalline form of valsartan, and most preferably is essentially free of crystalline forms of valsartan. “Essentially free of crystalline forms of valsartan” means that no crystalline forms of valsartan can be detected within the limits of a powder X-ray diffractometer.

The term “coprecipitate” means a solid composite comprising amorphous valsartan homogeneously dispersed in a pharmaceutically acceptable carrier, which is also in an amorphous state. The ratio of valsartan to a carrier in weight to weight is from 1% to 600%, preferably 10% to 300%, more preferably 50% to 200%, and most preferably 80% to 120%.

The term “carrier” in this invention can be any pharmaceutically acceptable excipients; preferably carriers are selected from polyvinylpyrolidone (PVP), crosslinked-polyvinylpyrolidone, polyvinylpyrolidone vinyl acetate copolymer (PVP-VA64) and solid polyethylene glycol (PEG).

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical formulation” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions of the present invention encompass any composition made by admixing the active ingredient, active ingredient dispersion or composite, additional active ingredient(s), and pharmaceutically acceptable excipients.

According to one aspect of the invention, there is new coprecipitate of amorphous valsartan with a pharmaceutically acceptable carrier. The said coprecipitate can be obtained through distillation and spray-drying techniques. The obtained coprecipitate in the invention is a solid, homogeneous, stable and amorphous composite with good material flowing property and suitable for preparation of pharmaceutical formulation such as solid dosage forms.

Comparing to the adsorbates of valsartan described in US2005/0234016, the novel coprecipitate of the current invention has following advantages. In coprecipitate, the amorphous valsartan is homogenously dispersed on the amorphous carrier. This highly dispersed, homogenous and amorphous solid composite does not contain crystalline carrier and therefore the amorphous active ingredient, valsartan, will not be converted back into crystalline forms, and is in fact stabilized in such coprecipitate. Whereas adsorbates of valsartan contain the crystalline carrier, which can induce the conversion of amorphous valsartan into the crystalline material, resulting in the change of physical and chemical properties of the active ingredient.

According to the invention, the coprecipitate of amorphous valsartan with pharmaceutically acceptable carrier contains less than 15% crystalline valsartan, preferably less than 10% crystalline valsartan, more preferably less than 5% crystalline valsartan, and most preferably less than 2% crystalline valsartan or essentially (less than 0.5%) no crystalline valsartan. At mean time, the coprecipitate contains less than 15% crystalline carrier, preferably less than 10% crystalline carrier, more preferably less than 5% crystalline carrier, and most preferably less than 2% crystalline carrier or essentially (less than 0.5%) no crystalline carrier.

A coprecipitate of amorphous valsartan with amorphous pharmaceutically acceptable carrier is obtained in the form of white powders after spray-drying processing or distillation processing under reduced pressure.

During the process of preparing novel coprecipitate of amorphous valsartan with a pharmaceutically acceptable carrier, particles sizes are reduced into molecular level, and to what can result in an enhanced dissolution rate due to both increase in the surface area and solubilization.

The present invention produces compositions of a pharmaceutically acceptable carrier in which valsartan is stabilized in its amorphous form. The reduced size of particles and quality of coprecipitate provide for a high surface area of active compound what may result in improved bioavailability.

According to the invention, an appropriate ratio of valsartan to a pharmaceutically acceptable carrier, ranges from 0.1:1 to 1:0.1 parts by weight, preferably from 0.1:1 to 1:0.5, more preferably from 1:1 to 1:2.

Pharmaceutically acceptable carriers for preparing amorphous coprecipitate of the invention can be any materials described in above cited Encyclopedia of Pharmaceutical Technology (Vol. 3, Table 1 on page 345), preferably polyvinylpyrolidone (PVP), crosslinked-polyvinylpyrolidone (crospovidone), polyvinylpyrolidone vinyl acetate copolymer (PVP-VA64).

The average molecular weight of polyvinylpyrrolidone (PVP) is not critical and any average molecular weight of PVP (see e.g. Handbook of Pharmaceutical Excipients, 3^{nd} Ed (2000), 433-439, American Pharmaceutical Association Washington and The Pharmaceutical Press London), may be used, but preferably PVP ranges from 10,000 to 100,000 (K=17-96), most preferably PVP with K=30, because the capability of preventing crystallization of valsartan and the solubility in the solvent are well balanced.

The crospovidone (or cross-polyvinylpyrrolidone, cross-PVP) described in reference of Handbook of Pharmaceutical Excipients, 3^{nd} Ed (2000), 163-164, American Pharmaceutical Association Washington and The Pharmaceutical Press London) may be used.

The average molecular weight of polyvinylpyrrolidone/vinylacetate copolymer (PVP-VA64 or PVP-VA or copolyvidone) is also not critical and any average molecular weight of PVP-VA64 can be used, but preferably, PVP-VA64 with K value 24-36 should be used. PVP VA 64 is water-soluble vinylpyrrolidone-vinyl acetate copolymer contains the two components in a ratio of 6:4. Because of its vinyl acetate component, PVP VA 64 is somewhat more hydrophobic, less hygroscopic and has greater elasticity than PVP.

The amount percentage of crystalline form in amorphous valsartan is usually determined by X-ray powder diffraction pattern method (X-RPD). Due to lack of structure order, the amorphous valsartan in its X-RPD spectrum does not show any dispersible peaks, only display a broad diffraction. In addition, amorphous material in differential scanning calorimetry (DSC) does not show any endothermic or exothermic peaks. Therefore, X-RPD and DSC techniques can be used to confirm the amorphous nature of the obtained product.

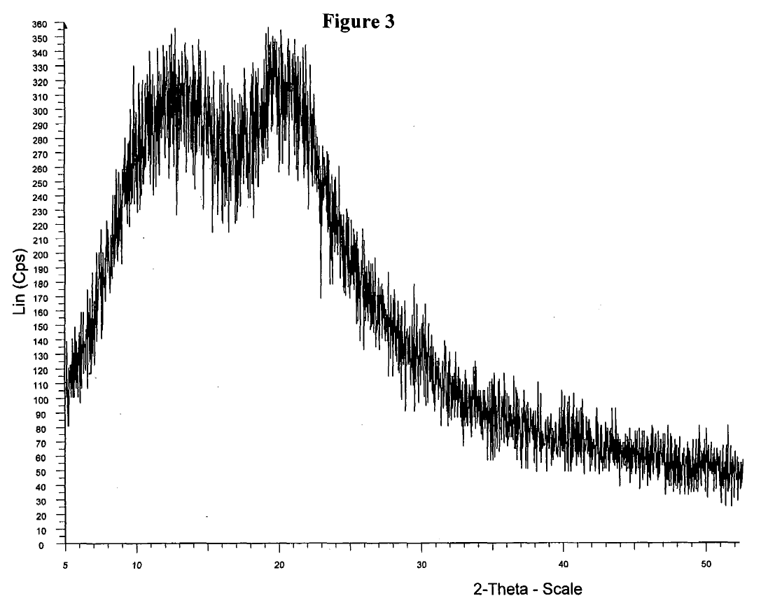

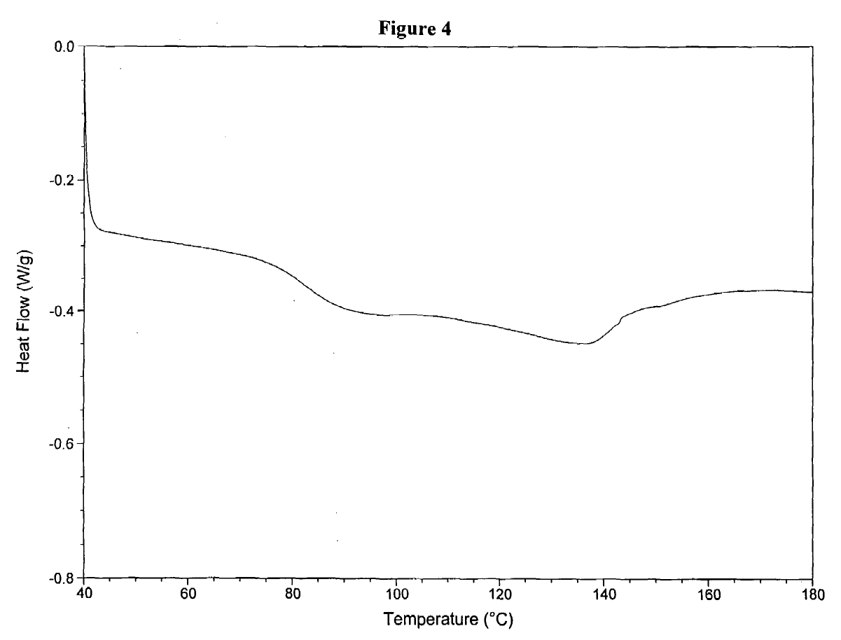

Similarly, the amount percentage of crystalline form in coprecipitate of amorphous valsartan with a pharmaceutically acceptable carrier can also determined by X-ray powder diffraction pattern method (X-RPD). Due to lack of structure order, the amorphous valsartan and carrier in X-RPD spectrum does not show any dispersible peaks, only display a broad line (see FIG. 3). In addition, amorphous material in differential scanning calorimetry (DSC, see FIG. 4) does not show any endothermic or exothermic peaks. Therefore, X-RPD and DSC techniques can be used to confirm the amorphous nature of the obtained coprecipitate.

In another aspect, the current invention provides a process for preparing the coprecipitate of amorphous valsartan with pharmaceutically acceptable carrier (distillation under reduced pressure), including following steps: a) dissolving the amorphous or mixture of amorphous and crystalline valsartan and a carrier in alcohol solvents; b) stirring the solution until it becomes clear; c) removing the solvent by distillation under reduced pressure; d) grinding the product, further drying the product under vacuum at elevated temperature until loss of drying is less than 0.5% or constant.

Specifically, the mixture of amorphous and crystalline valsartan and carrier is usually dissolved in acetone or alcohol solvents, such as methanol or ethanol. The concentration of valsartan is from 1% to 20% (w/v, thereafter), preferably from 2% to 15%. The concentration of carrier is from 1% to 50% (w/v), preferably from 2% to 30%. The weight ratio of active ingredient to carrier is 1:10 to 10:1, preferably 1:2 to 5:1, more preferably 1:1 to 2:1. The solution is heated and then the solvent is removed under the reduced pressure until dryness to obtain the solid product. The reduced pressure means that the pressure is less than 400 mmHg, preferably less than 100 mmHg, and more preferably less than 30 mmHg. The obtained product is grounded, dried under vacuum at elevated temperature. The drying temperature is preferably from 20 to 70° C. The drying time is from 12 hours to 48 hours. Most preferably, the drying temperature is 35 to 40° C. and drying time is 18-24 hours. The dried product is further grounded or sieved, and then dried under 35-40° C. for another 8-10 hours or until the loss of drying is less than 0.5% or constant.

We discovered that novel coprecipitate of amorphous valsartan with pharmaceutically acceptable carrier can be obtained by this simple and reproducible process.

According to a process of the invention, the alcohol solvents are selected from acetone, methanol, ethanol, propanol, isopropanol or branched or straight chain butynol, preferably methanol, ethanol or isopropanol or their mixture of any two solvents.

According to a preferred process of the invention, the coprecipitate can be obtained by following procedure: dissolving the starting material in acetone or alcohol solvent or their mixture of any two solvents, removing the solvents under the reduced pressure until dryness, grinding the obtained product, and finally drying the product under vacuum at 35-45° C.

In a further aspect, the current invention provides a spray-drying process for preparing the coprecipitate of amorphous valsartan with pharmaceutically acceptable carrier, including following steps: a) dissolving the amorphous or mixture of amorphous and crystalline valsartan and carrier in water acetone, or alcohol solvent or their mixture of any two solvents thereof; b) stirring the solution until it becomes clear; c) removing the solvent by spray-drying; d) further drying the product under vacuum at elevated temperature until loss of drying is less than 0.5% or constant.

The spray drying process can be carried out using any commercially available dryers, which are used, operates on the principle of nozzle spraying in a parallel flow. For instance, the sprayed product and drying gas flow in the same direction. The drying gas can be air or inert gasses such as nitrogen, argon and carbon dioxide. Nitrogen gas is preferred in this invention. For valsartan and carrier solution, the spray drying in-let temperature is about 140-180° C., and the out-let temperature is about 90-60° C. at a feed rate of 5-25 ml/min.

Specifically, the amorphous or mixture of amorphous and crystalline valsartan and carrier are usually dissolved in water, acetone or alcohol solvents, such as methanol or ethanol. The concentration of valsartan is from 1% to 20% (w/v), preferably from 2% to 15%. The concentration of carrier is from 1% to 50% (w/v), preferably from 2% to 30%. The weight ratio of active ingredient to carrier is 1:10 to 10:1, preferably 1:2 to 5:1, more preferably 1:1 to 2:1. If necessary, the solution can be heated to completely dissolve the starting materials, and then the solvent is removed by spray drying to obtain the solid product. The solution is cooled to 30° C., and then proceeds with spray drying.

The product obtained from spray drying is further dried to remove the solvent. The product can be dried in a tray drier or dried under vacuum or in a Fluid Bed Dryer. The drying temperature is preferably from 20 to 70° C., drying time is preferably from 8-24 hours. The most preferred drying temperature 35-40° C. and drying time is 12 to 15 hours. After drying, the obtained solid product is the coprecipitate of amorphous valsartan with a carrier.

We found that homogenous coprecipitate of amorphous valsartan with pharmaceutically acceptable carrier can be obtained using this simple and reproducible spray drying process.

According to a process of the invention, the starting material of valsartan can be obtained by any methods described in the prior art such as U.S. Pat. No. 5,399,578 or WO02/06253. The starting material valsartan can be crude or pure valsartan, including any solvates or hydrates, preferably purity is more than 95%, more preferably purity is more than 98%, most preferably purity is more than 99%. Starting material valsartan can be any polymorph forms, including amorphous or crystalline form or their mixture thereof. With the processes where valsartan goes into solution, the form of the starting material is of minimal relevance since any solid-state structure is lost in solution.

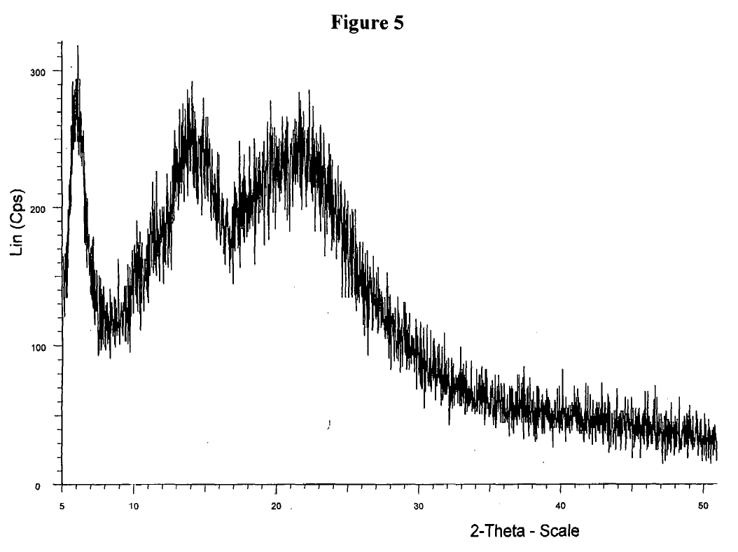

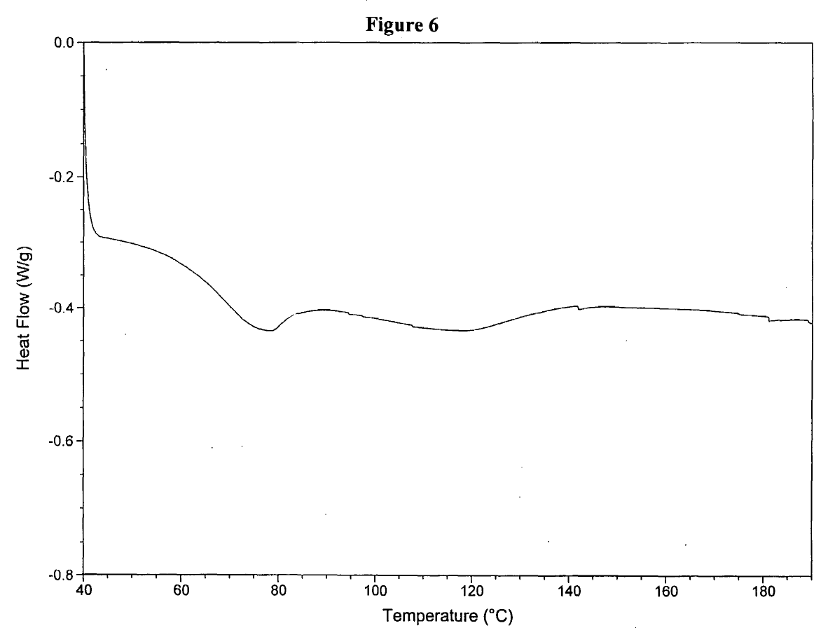

According to a process of the invention, the amorphous nature of coprecipitate of amorphous valsartan with a pharmaceutically acceptable carrier can also be determined by X-ray powder diffraction pattern method (X-RPD), as shown in FIG. 3 or 5. Due to lack of structure order, the amorphous valsartan and carrier in X-RPD spectrum does not show any dispersible peaks, only display a broad diffraction (see FIG. 3 or 5). In addition, amorphous nature of the coprecipitate can also be determined by differential scanning calorimetry (DSC, see FIG. 4 or 6), which does not show any endothermic or exothermic peaks. Therefore, X-RPD and DSC techniques can be used to confirm the amorphous nature of the obtained coprecipitate.

The coprecipitate of amorphous valsartan with a pharmaceutically acceptable carrier, plus additional excipients can be further used to make pharmaceutical formulation. Therefore, the current invention provides a pharmaceutical formulation comprising an effective amount of amorphous valsartan with a pharmaceutically acceptable carrier and other excipients.

Pharmaceutical formulation can be unit dosage form, and can be oral or parental dosage forms, which may be in the form of suspensions, solutions, elixirs or stolid dosage forms, e.g. tablets, capsules, parenteral dosage forms, comprising co-precipitate of amorphous valsartan with a pharmaceutically acceptable carrier and other suitable excipients. Other excipients may be included in the pharmaceutical formulations to further improve the stabilization and/or de-agglomeration of the amorphous particles of active substance. A preferred oral solid dosage form is a tablet or capsule.

Thus, the coprecipitate of the present invention can be administered by injection, that is, intravenously, intramuscularly, intracutaneously, subcutaneously, intraduodenally, or intraperitoneally. Also, the coprecipitate of the present invention can be administered by inhalation, for example, intranasally. Additionally, the coprecipitate of the present invention can be administered transdermally. It will be obvious to those skilled in the art that the following dosage forms may comprise as the active component, either coprecipitate, or a corresponding pharmaceutically acceptable salt of a compound of the present invention.

For preparing pharmaceutical formulation from solid coprecipitate of the present invention, pharmaceutically acceptable excipients can be either solid or liquid.

Solid pharmaceutical formulation includes powders, tablets, pills, capsules, cachets, suppositories, and dispersible granules. A solid excipient can be one or more substances that may also act as diluents, flavoring agents, solubilizers, lubricants, suspending agents, binders, preservatives, tablet disintegrating agents, or an encapsulating material.

In powders, the excipient is a finely divided solid that is in a mixture with the finely divided active component. In tablets, the active component is mixed with the carrier having the necessary binding properties in suitable proportions and compacted in the shape and size desired.

The powders and tablets preferably contain from one or ten to about seventy percent of the active compound. Suitable carriers are magnesium carbonate, magnesium stearate, talc, sugar or lactose, pectin, dextrin, starch, gelatin, tragacanth, methylcellulose, sodium carboxymethylcellulose, a low melting wax, cocoa butter, and the like. Similarly, cachets and lozenges are included. Tablets, powders, capsules, pills, cachets, and lozenges can be used, as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, retention enemas, and emulsions, for example water or water propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizing, and thickening agents as desired.

Also included are solid form formulations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical formulation is preferably in unit dosage form. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 1 mg to 7000 mg, preferably 5 mg to 2000 mg, more preferably 5 to 500 mg according to the particular application and the potency of the active component. The pharmaceutical formulation can, if desired, also contain other compatible therapeutic agents. In particular, the pharmaceutical formulation can contain both valsartan and diuretic agent such as hydrochlorothiazide as active ingredients. However, the optimum dosage for the individual subject being treated will be determined by the person responsible for treatment, generally smaller doses being administered initially and thereafter increments made to determine the most suitable dosage.

In a preferred aspect of the invention, the pharmaceutical formulation conveniently contains about 1-1000 mg, preferably 20-400 mg of valsartan as active ingredient, wherein valsartan is provided as a said coprecipitate.

In another preferred aspect of the invention, the pharmaceutical formulation contains almost 20-400 mg valsartan and 2-40 mg hydrochlorothiazide as active ingredients, wherein valsartan is provided as a said coprecipitate.

A further aspect of the invention relates to a novel stable solid solution of valsartan with a suitable pharmaceutically acceptable carrier. Pharmaceutical acceptable carrier for preparing solid solutions of the invention is preferably polyethylene glycols (PEG), from 4000 to 20,000 of average molecular weight, more preferably PEG 4000-8000.

A process for the preparation of a solid solution comprising the steps: a) melting valsartan and a pharmaceutically acceptable carrier to form a melt; b) cooling the obtained melted solution. Said novel solid solution of the invention can be used for the preparation of pharmaceutical formulation, e.g. solid dosage forms, preferably tablets, which further comprise other suitable excipients for use in the treatment and/or prophylaxis of cardiovascular diseases and hypertension, conditions associated with cardiovascular diseases and hypertension and certain complications thereof.

We have surprisingly and unexpectedly found that preparing pharmaceutical compositions comprising adsorbates of a crystalline pharmaceutically acceptable carrier homogeneously integrated with an amorphous form of an active agent as described in US2005/0234016 may be avoided. Instead of the preparation of adsorbates, co-precipitates and solid solution comprising amorphous pharmaceutically acceptable carriers homogeneously dispersed with an amorphous form of valsartan can be prepared by using different materials and processes from those used in publication US2005/0234016. Said novel coprecipitates and solid solution of the invention can be used for the preparation of pharmaceutical formulations, e.g., solid dosage forms, preferably tablets, which further comprises other suitable excipients, or in combination with the duretic hydrochlorothiazide for use in the treatment of cardiovascular diseases and hypertension or conditions associated with cardiovascular diseases and hypertension and certain complications thereof.

Having thus described the invention with reference to particular preferred embodiments, those in the art can appreciate modifications to the invention as described and illustrated that do not depart from the spirit and scope of the invention as disclosed in the specification. The following examples are set to illustrate the invention, and aid to understanding the invention, but not intended to, and should not be construed to limit its scope in any way.

### EXAMPLES

### Example 1

### Preparation of Mixture of Amorphous and Crystalline Valsartan

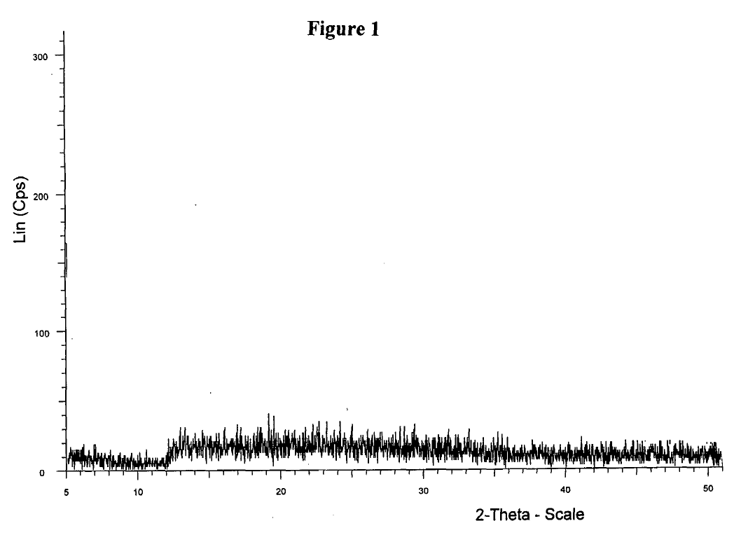

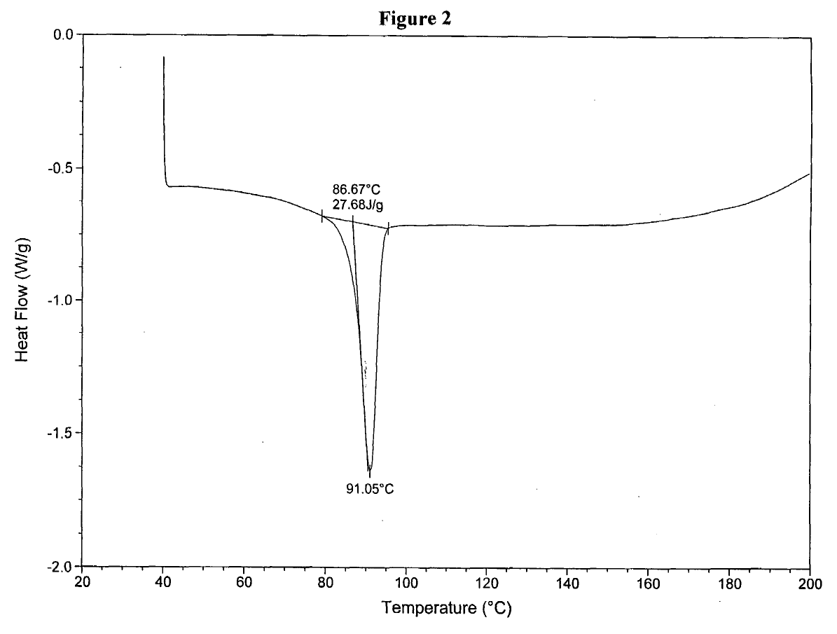

Crude valsartan (10 g, obtained by the procedure described in Example 16 of U.S. Pat. No. 5,399,578) was dissolved in methyl propyl ketone (50 ml) at ambient temperature, and suspension mixture was heated to 50° C. to obtain a clear solution. To the solution was then slowly added Hexane (40 mL), and cooled the solution to an ambient temperature. The solution was kept aside for about 1 hour to crystallize the solid mass. The product was then isolated by filtration and dried at 50-50° C. to constant weight to obtain 6.5 g solid product (yield 65%). The powder X-ray diffractogram (FIG. 1) and DSC (FIG. 2) of the obtained product showed that the resulting substance contained most amorphous material, but with some crystalline valsartan can be clearly detected.

### Example 2

### Preparation of Coprecipitate of Valsartan with Polyvinylpyrolidone (PVP)

Method A: Valsartan (3 g, obtained by the procedure described in Example 16 of U.S. Pat. No. 5,399,578) and 9.0 g polyvinylpyrolidone (PVP, K=30) was dissolved in ethanol (150 ml) at ambient temperature, and suspension mixture was heated to 50° C. to obtain a clear solution. The solvent was evaporated through distillation under vacuum (30-80 mm Hg) at about 40° C. to about 70° C. The product was then isolated (about 11 g) when no visible liquid was remained and drying was continued under vacuum at about 40° C. for 24-48 hours to remove the solvent. The powder X-ray diffractograrm and DSC of the solid coprecipitate showed that the resulting substance was amorphous coprecipitate.

Method B: Valsartan (5 g, obtained from Example 1 of this invention) and 5.0 g polyvinylpyrolidone (PVP, K=30) was dissolved in acetone (100 ml) at ambient temperature, and suspension mixture was heated to 50° C. to obtain a clear solution. The solvent was evaporated through distillation under vacuum (30-80 mm Hg) at about 40° C. to about 70° C. The product was then isolated (about 8.5 g) when no visible liquid was remained, and dried under vacuum at about 40° C. for 24-48 hours to remove the solvent. The powder X-ray diffractograrm and DSC showed that the resulting substance was amorphous coprecipitate.

Method C: Valsartan (5 g, obtained by the procedure described in Example 16 of U.S. Pat. No. 5,399,578) and 4.0 g polyvinylpyrolidone (PVP, K=30) was dissolved in ethanol (150 ml) at ambient temperature, and suspension mixture was heated to 50° C. to obtain a clear solution. The solution was cooled to 35° C., and then subjected to spray drying in a Mini-Spray Dryer (e.g., Buchi Model-190) at an inlet temperature 145-175° C. and outlet temperature 70-90° C. using nitrogen gas. The light-white fine powder in an amorphous form was obtained. The product was further dried under vacuum at about 40° C. for about 24 hours to afford 7.5 g of the desired solid coprecipitate product, which is amorphous material as characterized by powder X-ray diffractogram or DSC.

Method D: Valsartan (5 g, obtained by the procedure described in Example 16 of U.S. Pat. No. 5,399,578) and 6.0 g polyvinylpyrolidone (PVP, K=30) was dissolved in 150 mL of ethanol and water (1:1, v/v) at ambient temperature, and suspension mixture was heated to 50° C. to obtain a clear solution. The solution was cooled to 35° C., and then subjected to spray drying in a Mini-Spray Dryer (e.g., Buchi Model-190) at an inlet temperature 145-175° C. and outlet temperature 70-90° C. using nitrogen gas. The product obtained was further dried under vacuum at about 40° C. for about 24 hours to afford 9.5 g of the desired solid coprecipitate product, which is amorphous material as characterized by powder X-ray diffractogram or DSC.

### Example 3

### Preparation of Coprecipitate of Valsartan with Polyvinylpyrolidone Vinyl Acetate Copolymer (PVP-VA64)

Method A: Valsartan (5 g, obtained by the procedure described in Example 16 of U.S. Pat. No. 5,399,578) and 8.0 g polyvinylpyrolidone vinyl acetate copolymer (PVP-VA64, Plastone S-630, K=26-34)) was dissolved in ethanol (150 ml) at ambient temperature, and suspension mixture was heated to 50° C. to obtain a clear solution. The solvent was evaporated through distillation under vacuum (30-80 mm Hg) at about 40° C. to about 70° C. The product was then isolated (about 11.5 g) when no visible liquid was remained, and dried under vacuum at about 40° C. for 24-48 hours to remove the solvent. The powder X-ray diffractograrm and DSC showed that the resulting substance was amorphous coprecipitate.

Method B: Valsartan (5 g, obtained from example 1 of this invention) and 5.0 g polyvinylpyrolidone vinyl acetate copolymer (PVP-VA64, Plastone S-630, K=26-34) was dissolved in acetone (100 ml) at ambient temperature, and suspension mixture was heated to 50° C. to obtain a clear solution. The solvent was evaporated through distillation under vacuum (30-80 mm Hg) at about 40° C. to about 70° C. The product was then isolated (about 8.7 g) when no visible liquid was remained, and dried under vacuum at about 40° C. for 24-48 hours to remove the solvent. The powder X-ray diffractograrm and DSC showed that the resulting substance was amorphous coprecipitate.

Method C: Valsartan (5 g, obtained by the procedure described in Example 16 of U.S. Pat. No. 5,399,578) and 4.0 g polyvinylpyrolidone vinyl acetate copolymer (PVP-VA64, Plastone S-630, K=26-34)) was dissolved in ethanol (150 ml) at ambient temperature, and suspension mixture was heated to 50° C. to obtain a clear solution. The solution was cooled to 35° C., and then subjected to spray drying in a Mini-Spray Dryer (e.g., Buchi Model-190) at an inlet temperature 145-175° C. and outlet temperature 70-90° C. using nitrogen gas. The light-white fine powder of valsartan and PVP-VA64 in an amorphous form was obtained. The product was further dried under vacuum at about 40° C. for about 24 hours to afford 8.2 g of the desired solid coprecipitate product, which is amorphous material as characterized by powder X-ray diffractogram or DSC.

Method D: Valsartan (5 g, obtained by the procedure described in Example 16 of U.S. Pat. No. 5,399,578) and 6.0 g polyvinylpyrolidone vinyl acetate copolymer (PVP-VA64, Plastone S-630, K=26-34)) was dissolved in 150 mL of acetone and water (1:1, v/v) at ambient temperature, and suspension mixture was heated to 50° C. to obtain a clear solution. The solution was cooled to 35° C., and then subjected to spray drying in a Mini-Spray Dryer (e.g., Buchi Model-190) at an inlet temperature 145-175° C. and outlet temperature 70-90° C. using nitrogen gas. The product obtained was further dried under vacuum at about 40° C. for about 24 hours to afford 9.3 g of the desired solid coprecipitate product, which is amorphous material as characterized by powder X-ray diffractogram or DSC.

### Example 4

### Preparation of Coprecipitate of Valsartan with Crospolyvinylpyrolidone

Method A: Valsartan (4 g, obtained by the procedure described in Example 16 of U.S. Pat. No. 5,399,578) and 6.0 g cross-linked polyvinylpyrolidone (cross-PVP, Polyplastone XL) was dissolved in ethanol (150 ml) at ambient temperature, and suspension mixture was heated to 60° C. to obtain a clear solution. The solvent was evaporated through distillation under vacuum (30-80 mm Hg) at about 40° C. to about 70° C. The product was then isolated (about 8 g) when no visible liquid was remained, and dried under vacuum at about 40° C. for 24-48 hours to remove the solvent. The powder X-ray diffractograrm and DSC showed that the resulting substance was amorphous coprecipitate.

Method B: Valsartan (5 g, obtained by the procedure described in Example 16 of U.S. Pat. No. 5,399,578) and 4.0 g cross-linked polyvinylpyrolidone (cross-PVP, Polyplastone XL) was dissolved in ethanol (150 ml) at ambient temperature, and suspension mixture was heated to 50° C. to obtain a clear solution. The solution was cooled to 35° C., and then subjected to spray drying in a Mini-Spray Dryer (e.g., Buchi Model-190) at an inlet temperature 145-175° C. and outlet temperature 70-90° C. using nitrogen gas. The product was further dried under vacuum at about 40° C. for about 24 hours to afford 7.5 g of the desired solid coprecipitate product, which is amorphous material as characterized by powder X-ray diffractogram or DSC.

### Example 5

### Preparation of Solid Solution of Valsartan with Solid PEG

Method A: 5 g of PEG 4000 (polyethylene glycol 4000) and 5 g of valsartan (obtained from example 1 of this invention) were mixed and the resulted mixture was heated to 80° C. The obtained solution was then allowed to cool to room temperature and solid mass of solid solution of valsartan with PEG 4000 was obtained; its amorphous nature can be characterized by X-RPD and DSC.

Method B: 5 g of PEG 6000 (polyethylene glycol 6000) and 5 g of valsartan (obtained from example 1 of this invention) were mixed and the resulted mixture was heated to 82° C. The obtained solution was then allowed to cool to room temperature and solid mass of solid solution of valsartan with PEG 6000 was obtained; its amorphous nature can be characterized by X-RPD and DSC.

### Example 6

### Preparation of Pharmaceutical Formulation Containing Coprecipitate

Pharmaceutical formulation suitable for tablet dosage forms made using coprecipitate of amorphous valsartan with PVP and other excipients are described in Table 1. As shown in Table 1, formula A provide a dose of 40 mg valsartan in a 200 mg tablet, formula B provides a dose of 80 mg valsartan in a 250 mg tablet, and formula C provides a dose of 80 mg valsartan and 12.5 mg hydrochlorothiazide in a 250 mg tablet.

There were two major steps involved in manufacturing the tablets: (1) preparation of valsartan tablet core; (2) coating the tablet core.

TABLE 1
Formulation for Valsartan Tablet Comprising Coprecipitate

| | mg/tablet | | |
|---|---|---|---|
| Component | Formula A | Formula B | Formula C |
| Coprecipitate of amorphous valsartan | 80 | 160 | 160 |
| with PVP (1:1, w/w) | | | |
| Hydrochlorothiazide | — | — | 12.5 |
| Microcrystalline cellulose (Avicel | 70 | 50 | 37.5 |
| PH-101/102) | | | |
| Anhydrous lactone | 30 | 20 | 20 |
| Sodium starch glycolate | 10 | 10 | 10 |
| Magnesium stearate | 5 | 5 | 5 |
| Coating agent(opadry white), water* | 5 | 5 | 5 |
| Total | 200 | 250 | 250 |

*Removed during the coating process

The coprecipitate and following ingredients were sifted through a clean screen (typically 0.066″): anhydrous lactose, sodium starch glycolate and microcrystalline cellulose. The screened materials were transferred into a high shear (high-energy) mixer and blended for ten (10) minutes at 100 rpm. The blended material was compressed on a Kikusui Libra tablet compression machine to a target weight of 200 mg for the 40 mg tablets, and 250 mg for 80 mg tablets.

The tablet cores were then transferred to a tablet-coating machine (pan coater). The tablet bed was pre-heated with warm air (approximately 60° C.). The pan speed was adjusted to 5-9 RPM before starting the spray cycle. The spray cycle was activated. The exhaust temperature was maintained between 40° C. and 50° C. throughout the cycle. After the proper amount of solution was applied, the coated tablets were dried for approximately two (2) minutes. Steps were repeated for all pans to coat all tablets in the batch and film coated until the tablet weight has increased by 2.0% to 4.5%. All tablets were packaged in plastic bottles with desiccants, and the bottles were heat sealed, then placed under the storage conditions.

The obtained tablets were packaged into HDPE bottle and induction-sealed, and then stressed under 40° C./75% for three months. The stressed tablets were analyzed by a HPLC method. The results indicated that the total degradation products of valsartan were less than 2.0%. In addition, the X-ray powder diffraction experiments demonstrated that no crystalline valsartan was detected. The quality and performance of drug product as measured by dissolution was also unchanged. Therefore the drug product comprising coprecipitate of amorphous valsartan with a carrier are stable, and it has an adequate shelf life under normal process and storage conditions.

Details of HPLC method were: column: Zorbax SB-CN C18, 250 mm×4.6 mm, 5 um; mobile phase A is 15% acetonitrile/85% water (0.05% TFA) and mobile phase B is 85% acetonitrile/water (0.05% TFA); holding mobile phase A for 5 min, starting the gradient from 5 min to 25 min, B from 0% to 30%, from 25 min to 50 min, B from 30% to 100%, and then holding 100% B from 50 min to 60 min, reconditioning column with mobile phase A. UV detection wavelength was 250 nm.

While the present invention has been described in terms of its specific embodiments, certain modifications and equivalents will be apparent to those skilled in the art and are intended to be included within the scope of the present invention.

We claim:

1. A coprecipitate of amorphous valsartan with a pharmaceutically acceptable carrier, wherein the weight ratio of amorphous valsartan to the carrier ranges from 1:0.1 to 0.1:1.

2. The coprecipitate of amorphous valsartan with a pharmaceutically acceptable carrier of claim 1, wherein its X-ray powder diffraction pattern displays one or more broad diffuse halos and lacks any discernible peaks and its differential scanning calorimetry lacks any endothermic or exothermic peaks.

3. The coprecipitate of amorphous valsartan with a pharmaceutically acceptable carrier of claim 1, wherein the carrier is selected from the group consisting of polyvinylpyrrolidone, cross-linked polyvinylpyrrolidone, polyvinylpyrrolidone vinyl acetate copolymer.

4. The coprecipitate according to claim 1, wherein it is the coprecipitate of amorphous valsartan with polyvinylpyrrolidone.

5. The coprecipitate according to claim 1, wherein it is the coprecipitate of amorphous valsartan with cross-linked polyvinylpyrrolidone.

6. The coprecipitate according to claim 1, wherein it is the coprecipitate of amorphous valsartan with polyvinylpyrrolidone vinyl acetate copolymer (PVP-VA64).

7. The coprecipitate according to claims 1, wherein the amount of crystalline valsartan is less than 15% by weight.

8. The coprecipitate according to claims 1, wherein the amount of crystalline valsartan is less than 5% by weight.

9. The coprecipitate according to claims 1, wherein the amount of crystalline valsartan is less than 2% by weight.

10. A process for the preparation of a coprecipitate of amorphous valsartan with a pharmaceutically acceptable carrier, comprising the steps of: a) dissolving valsartan in an organic solvent or in an aqueous solution of organic solvent, b) adding pharmaceutically acceptable carrier, c) removing the solvents by spray-drying or vacuum distillation, d) drying the obtained product.

11. The process according to claims 10, wherein a pharmaceutically acceptable carrier is selected from the group consisting of polyvinylpyrrolidone, cross-linked polyvinylpyrrolidone and polyvinylpyrrolidone vinyl acetate copolymer (PVP-VA64).

12. The process according to claims 10, wherein the weight ratio of amorphous valsartan to the carrier ranging from 1:0.1 to 0.1:1.

13. The process according to claims 10, wherein the weight ratio of amorphous valsartan to the carrier ranging from 1:1 to 1:2.

14. The process according to claims 10, wherein an organic solvent is selected from the group consisting of methanol, ethanol and acetone.

15. The process according to claims 10, wherein the ratio of organic solvent to water is from about 9:1 to about 1:1 (v/v).

16. The process according to claims 10, wherein the ratio of organic solvent to water is from about 9:1 to about 7:3 (v/v).

17. A pharmaceutical formulation comprising the coprecipitate of amorphous valsartan with a pharmaceutically acceptable carrier according to claim 1 and at least one additional excipient.

18. The pharmaceutical formulation according to claim 17, wherein the solid dosage form is tablets, capsules, powders, cachets, suppositories, or dispersible granules.
